(19) United States
(12) Patent Application Publication
(10) Pub. No.: US 2002/0122772 A1
(43) Pub. Date: Sep. 5, 2002

(54) Self foaming cleansing gel

(76) Inventors: Elvin Lukenbach, Flemington, NJ (US); Karine Dupre, Orleans (FR); Susan Niemiec, Yardley, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/905,387

(22) Filed: Jul. 13, 2001

Related U.S. Application Data
(60) Provisional application No. 60/218,483, filed on Jul. 14, 2000.

(51) Int. Cl. A61K 7/08; A61K 7/075

(52) U.S. Cl. 424/44; 424/70.24

(57) ABSTRACT

A self foaming composition comprising: (I) a surfactant mixture comprising (a) at least one anionic surfactant; (b) at least one amphoteric surfactant; and (c) optionally at least one nonionic surfactant; and (II) at least one self foaming agent. The composition is in the form of a liquid crystalline structure and wherein the ratio of (a) to (b) to (c) is selected such that when the surfactant composition is mixed with the self foaming agent a gel consistency is obtained. The cleansing composition is useful in the bath and the shower, as a facial cleanser, and as a shampoo.

### BACKGROUND OF THE INVENTION

1. Field of the Invention

The present invention relates to a composition that is useful for cleansing the body, face, and hair. The composition includes surfactants and a self foaming agent that causes the composition to foam upon contact with skin. The composition may be prepared such that a lamellar liquid crystal structure is formed.

2. Description of the Prior Art

Liquid body cleansers in the form of gels have recently replaced bar soaps in many consumers' showers and baths. Consumers generally equate foam with cleansing and a variety of other aesthetic attributes, therefore a high foaming body cleanser is highly desirable.

Conventional gel cleansing products do not foam significantly, compared with, for instance shampoos. This is because the shear process of skin cleansing does not introduce air in the quantity and bubble size in the same way as the array of hair fibers does in shampooing.

Another problem associated with conventional gel cleansing products is that the gels do not spread readily across the skin of the user. It is hypothesized that this is due to the physical structure of the composition. The spreadability of the gel may also be affected by thickeners, which typically are added to provide sufficient viscosity to the gel to prevent dripping.

It is known in the art that compositions containing water, surfactant, and oil may have different physical structures. Some physical structures the composition may take include a suspension or dispersion, an emulsion, a microemulsion, or a liquid crystal. The physical structure is affected by the surfactants used, the relative amounts of water, oil, and surfactant, and the process of preparing the composition.

Generally,speaking, droplets of either water or oil are created by the surfactants. The size of the droplets determines the physical structure of the composition. Relatively large droplets tend to create unstable emulsions, dispersions, or suspensions. Relatively small droplets (generally less than 1 micron) create stable microemulsions or liquid crystals. It is believed that the physical structure of the composition may affect the cleansing properties, such as foaming and spreading on the skin. The physical structure of the composition may also affect how the composition feels on the skin of the user.

World Patent Application No. WO 9703646 discloses a post foaming gel composition. The composition contains a base material of at least one detergent and a thickener, and a foaming material. At least part of the foaming material has a particle size large enough such that it is in suspension in the base material. The base material has a viscosity greater than 9,500 centipoise (“cps”). Because of the high viscosity of this composition, it is necessary to exert shear forces thereto in order to increase the production of foam. As discussed above, cleansing properties and aesthetic properties, such as the feel of the cleanser on the skin, may be affected by the physical structure of the composition and the thickeners added to increase the viscosity of the base material.

World Patent Application No. WO 0039273 discloses a packaged aqueous self-foaming liquid cleansing composition. The composition contains a surfactant system comprising anionic surfactant, amphoteric or zwitterionic surfactant and a hydrophobic component, and a post foaming agent. The composition is substantially free of soap, thickens on the addition of the post foaming agent to the base composition, and are in the form of microemulsions.

U.S. Pat. No. 4,772,427 discloses a stable post foaming gel composition. The gel contains from 60% to 75% water, from 3% to 23% anionic surfactant, from 1% to 24% ethoxylated fatty alcohol or fatty ester surfactant, from 2% to 4% isopropyl myristate, from 1% to 10% mono or disaccharide, and from 5% to 20% saturated aliphatic hydrocarbon foaming agent. The presence of ethoxylated fatty alcohol or fatty ester surfactant is known to promote the formation of an emulsion. However, emulsions are typically undesirable for gel cleansers because consumers prefer clear gel cleansers, and emulsions typically are cloudy.

Given the gel compositions known in the art, there remains a need for improved high foaming gel cleansing compositions that spread easily along the skin.

### SUMMARY OF THE INVENTION

We have discovered that it is possible to produce a high-foaming gel cleansing composition that not only spreads more easily along the skin but also produces more foam more quickly than other foaming gels known in the art. This is done by controlling the physical structure and the viscosity of the composition as well as by selecting specific surfactant combinations for the composition. In one aspect, the present invention provides a self foaming composition comprising: (I) a surfactant mixture comprising (a) at least one anionic surfactant; (b) at least one amphoteric surfactant; and (c) optionally at least one nonionic surfactant; and (II) at least one self foaming agent. The composition is in the form of a liquid crystalline structure and the ratio of (a) to (b) to (c) is selected such that when the surfactant composition is mixed with the self foaming agent a gel consistency is obtained.

### BRIEF DESCRIPTION OF THE FIGURES

FIG. 1 is an electron micrograph of the formulation produced in Example 2.

FIG. 2 is an electron micrograph of the formulation produced in Example 2 showing a cross sectional view.

### DETAILED DESCRIPTION OF PREFERRED EMBODIMENTS

The self-foaming cleansing composition includes a) an anionic surfactant b) an amphoteric surfactant, c) optionally a nonionic surfactant, and d) a self foaming agent. In one embodiment, the ratio of a to b to c is selected such that the surfactant composition mixture by itself has a viscosity less than about 9,500 cps, but when these surfactants are mixed with the self foaming agent, the viscosity of the final composition increases to the state of a gel, e.g., at least about 20,000 cps.

As discussed above, it is believed that the physical structure of the compositions according to the invention affect the cleansing properties, such as foaming and spreading on the skin. The physical structure of the composition may also affect how the composition feels on the skin of the user. Accordingly, the compositions according to the invention should have a liquid crystal structure, i.e., a lamellar liquid crystalline phase. A lamellar liquid crystalline phase is the stacking together of bilayers in exactly the same manner as one may visualize the formulation of surfactant crystal phases. The assembly of bilayers from pairs of molecules that are coupled head to head (or tail to tail) has been described for crystals. Once the bilayer is constructed, the bulk lamellar phase may be envisioned as resulting from the stacking of bilayers in the z-direction. The liquid crystalline structures can also be stacked in a hexagonal array of cylindrical stacked structures. Liquid crystalline structure may be used to dissolve substances that otherwise show limited solubility as described in Liquid Crystal Dispersions, Chapter 10, John West, pg. 349-371 in Technological Applications of Dispersions, Volume 52, Surfactant Science Series, ed. R. B. McKay, Marcel Dekker, Inc. New York, 1994. Thus, in the self foaming compositions of the present invention, the foaming agent is incorporated into the lamellar structures but the composition remains optically clear. The stacked bilayer array is believed to give the gel viscosity at rest, but makes the product easily spread on the skin, and as the lamellae are sheared, causes the incorporated foaming agent to be released to cause quick foaming action during use.

In contrast, to the lamellar structures of the self foaming compositions of the invention, microemulsions are optically isotropic transparent oil and water dispersions. Microemulsions are clear thermodynamically stable dispersions of two immiscible liquids with carefully adjusted emulsifier(s) (surfactants and cosurfactants) and are spherical in structure. Since the stacked bilayer structures are not present in microemulsions, the easy spreading characteristic is not present, and the quick release of the foaming agent on shear of the lamellae also is not to be expected. To make up for the deficiencies of microemulsions it is possible to add thickeners or viscosity modifiers to build viscosity. However, this solutionis undesirable as it compromises skin feel.

The self foaming compositions according to the invention generally comprise at least about 10%, preferably from about 10 to about 35%, based on the total weight of the composition, of the surfactant composition.

The first component of the surfactant compositionis one or more anionic surfactant. Preferably, the anionic surfactant is selected from the following classes of surfactants: alkyl sulfates; alkyl ether sulfates; alkyl monoglyceryl ether sulfates; alkyl monoglyceride sulfates; alkyl monoglyceride sulfonates; alkyl sulfonates; alkylaryl sulfonates; alkyl sulfosuccinates; alkyl ether sulfosuccinates; alkyl sulfosuccinates; alkyl amidosulfosuccinates; alkyl carboxylates; alkyl amidoethercarboxylates; alkyl succinates; fatty acyl sarcosinates; fatty acyl amino acids; fatty acyl taurates; fatty alkyl sulfoacetates; alkyl phosphates; alkyl ether phosphates; and mixtures thereof. A preferred anionic surfactant is sodium laureth sulfate.

The amount of anionic surfactant in the compositions of this invention may range, based upon the total weight of the composition, from about 2% to about 30%, preferably from about 5% to about 20%.

The second component of the surfactant compositionis one or more amphoteric surfactants. As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

Commercially available amphoteric surfactants are suitable for use in the present invention and include, but are not limited to amphocarboxylates, alkyl betaines, amidoalkyl betaines, amidoalkyl sultaines, amphophosphates, phosphobetaines, pyrophosphobetaines, carboxyalkyl alkyl polyamines, alkyl amino monoacetates, alkyl amino diacetates, and mixtures thereof. Betaine amphoteric surfactants are preferred. A particularly preferred betaine surfactant is cocamidopropyl betaine.

The amount of amphoteric surfactant in the compositions of this invention may range, based upon the final weight of the composition, from about 2% to about 20%, e.g. from about 3% to about 15% and from about 5% to about 10%.

An optional component of the surfactant composition is one or more nonionic surfactants. One class of nonionic surfactants useful in the present invention are polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester.

Examples of preferred polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G-4280.” Polysorbate 20, which is the laurate monoester of a mixture of sorbitol and sorbitol anhydrides condensed with approximately 20 moles of ethylene oxide, is available commercially from Uniqema Company under the tradename “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl gluocosides have about 1 to about 6 glucose residues per molecule of alkyl glucoside. Alkyl glucosides are the preferred nonionic surfactants. Suitable alkyl glucosides include, but are not limited to, octyl glucoside, decyl glucoside, and lauryl glucoside.

Additional nonionic surfactants that may be useful in this invention include: ethylene oxide/propylene oxide copolymers, (poly)glycerol esters and fatty acids, fatty acid alkanolamides, alkoxylated mono and di-alkanolamides, aminoxides, ethoxylated fatty alcohols and esters, fatty acid sucrose esters, ethoxylated glucosides, and fatty gluconamides.

In one embodiment of the present invention, the physical structure of the composition is a liquid crystal. Because ethoxylated fatty alcohol or fatty ester surfactants are known to promote the formation of an emulsion, these surfactants are not utilized when a liquid crystal is desired.

The amount of nonionic surfactant in the compositions of this invention may range, based upon the total weight of the composition, from about 1% to about 15%, e.g. from about 2% to about 10% or from about 3% to about 8%.

The fourth component of the present invention is a self foaming agent. As used herein, “self foaming agent” means any material that boils at least at the temperature of shower/bath water or the temperature of the human body. The self foaming agent may be selected from those known in the art, such as pentane, isopentane, butane, isobutane, etc. and mixtures thereof. A preferred mixture of self foaming agents is comprised of, based upon the total weight of self foaming agents, about 70% to about 90% isopentane and from about 10% to about 30% isobutane, e.g., about 85% isopentane and about 15% isobutane and about 75% isopentane and about 25% isobutane. The self foaming agent is present in the composition in an amount, based upon the total weight of the composition, from about 4% to about 15%, e.g. from about 6% to about 12% and from about 8% to about 10%.

The compositions of this invention may optionally contain one or more conditioning agents. Preferred cationic conditioning agents are selected from the following: a cationic cellulose derivative; a cationic guar derivative; and derivatives and copolymers of Diallyldimethylammonium chloride.

The amount of each conditioner component may range, based upon the total weight of the composition, from about 0.01 percent to about 1.0 percent, e.g. from about 0.01 percent to about 0.5 percent, and from about 0.01 to about 0.2 percent.

Preferably, the cationic cellulose derivative is a polymeric quaternary ammonium salt derived from the reaction of hydroxyethyl cellulose with a trimethylammonium substituted epoxide. The material known as Polyquaternium-10, commercially available from Amerchol Corporation of Edison, N.J. as “Polymer JR-400,” is especially useful in this regard.

The cationic guar derivative is preferably a guar hydroxypropyltrimonium chloride, available commercially from Rhodia Company under the tradename, “Jaguar C-17” and “Cosmedia Guar C261N” available from Cognis.

Other preferred cationic conditioning polymers are those derived from the monomer diallyldimethylammonium chloride. The homopolymer of this monomer is Polyquaternium-6, which is available commercially form Allied Colloids of Suffolk, Virginia under the tradename, “Salcare SC30.” The copolymer of diallyldimethylammonium chloride with acrylamide is known as Polyquaternium-7, and is also available from Allied Colloids under the tradename “Salcare SC10.”

The conditioner portion may be a combination of cationic cellulose derivative with a cationic guar derivative. In this embodiment, the cationic cellulose derivative is present in the composition in an amount, based on the overall weight of the composition, of from about 0.01 percent to about 2 percent, e.g. from about 0.05 percent to about 1.0 percent or from about 0.05 percent to about 0.3 percent, and the cationic guar derivative is present in an amount, based on the overall weight of the composition, of from about 0.01 percent to about 1.0 percent, e.g. from about 0.05 percent to about 1.0 percent or from about 0.05 percent to about 0.3 percent.

The conditioner portion may also be comprised of a cationic guar derivative and a homopolymer or copolymer of diallyldimethylammonium chloride. In this embodiment, the cationic guar derivative is present in an amount, based on the overall weight of the composition, from about 0.01 percent to about 0.5 percent, e.g. from about 0.01percent to about 0.2 percent, and the homopolymer or copolymer of diallyldimethylammonium chloride is present in an amount, based on the overall weight of the composition, from about 0.01 percent to about 0.5 percent, e.g, from about 0.01 percent to about 0.2 percent.

Skin conditioning agents such as glycerine and water insoluble hydrocarbon based skin conditioning emollients may also be useful in the present invention. Suitable water insoluble hydrocarbon based skin conditioning emollients include, but are not limited to, caprylic capric triglycerides, C₁₂-C₁₅ alcohols benzoate, and isopropyl palmitate. The water insoluble hydrocarbon based skin conditioning emollients may also be combined with other conditioners, for example cationic guar derivatives. The skin conditioning agents are generally present in the compositions of the invention at from about 0.01 to about 5.0 percent, preferably at from about 1.5 to 2 percent by weight based on the total weight of the composition.

The compositions of the present invention may also include one or more optional ingredients nonexclusively including foam boosters, a thickening agent, secondary conditioners, humectants, chelating agents, and additives which enhance their appearance, feel and fragrance, such as colorants, fragrances, preservatives, pH adjusting agents, and the like. Generally, the pH of the compositions of this invention is preferably maintained in the range of from about 3 to about 10, preferably from about 4 to about 7.5, and more preferably from about 5.0 to about 7.0.

Commercially available thickening agents that are capable of imparting the appropriate viscosity to the compositions may be suitable for use in this invention, but preferably are not utilized. In one embodiment using a thickener, the thickener should be present in the compositions in an amount sufficient to raise the Brookfield viscosity of the surfactant mixture without the self foaming agent to a value of between about 500 to about 9,500 cps. Examples of suitable thickening agents nonexclusively include: mono or diesters of 1) polyethylene glycol of formula HO—(CH₂CH₂O),H wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Illinois or from Comiel, S.p.A. of Bologna, Italy under the tradename, “PEG 6000 DS”.

When using a thickener component, it is also preferable to preblend the desired thickener with from about 5 percent to about 20 percent, based upon the total weight of the composition, of water and preferably at a temperature of from about 60° C. to about 80° C.

Commercially available secondary conditioners, such as volatile silicones, may be suitable for use in this invention. Preferably, the volatile silicone conditioning agent has an atmospheric pressure boiling point less than about 220° C. The volatile silicone conditioner may be present in an amount, based upon the total weight of the composition, from about 0 percent to about 3 percent, e.g. from about 0.25 percent to about 2.5 percent or from about 0.5 percent to about 1.0 percent. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Michigan under the tradename, “DC-345” and mixtures thereof, and preferably include cyclomethicone fluids.

Commercially available humectants, which are capable of providing moisturization and conditioning properties to the composition, are suitable for use in the present invention. The humectant is present in an amount, based upon the total weight of the composition, from about 0 percent to about 10 percent, e.g. from about 0.5 percent to about 5 percent or from about 0.5 percent to about 3 percent. Examples of suitable humectants nonexclusively include: 1) water soluble liquid polyols selected from the group comprising glycerine, propylene glycol, hexylene glycol, butylene glycol, dipropylene glycol, and mixtures thereof; 2)polyalkylene glycol of the formula HO—(R″O)_{b}—H wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH wherein c is an integer from about 5 to about 25; 4) urea; and mixtures thereof, with glycerine being the preferred humectant.

Examples of suitable chelating agents include those which are capable of protecting and preserving the compositions of this invention. Preferably, the chelating agent is ethylenediaminetetracetic acid (“EDTA”), and more preferably is tetrasodium EDTA, available commercially from Dow Chemical Company of Midland, Mich. under the tradename, “Versene 100XL” and is present in an amount, based upon the total weight of the composition, from about 0 to about 0.5 percent, e.g. from about 0.05 percent to about 0.25 percent.

Suitable preservatives include, but are not limited to, Quaternium-15, available commercially as “Dowicil 200” from the Dow Chemical Corporation of Midland, Mich., and sodium benzoate, and are present in the composition in an amount, based upon the total weight of the composition, from about 0 to about 0.2 percent, e.g. from about 0.05 percent to about 0.10 percent.

The compositions of this invention may be produced by first making a surfactant mixture, which contains all of the ingredients of the composition except for the self foaming agent. The surfactant mixture may be prepared by combining the surfactants and optional ingredients under ambient conditions by any conventional mixing means well known in the art, such as a mechanically stirred propeller, paddle, and the like. Although the order of mixing is not critical, it is preferable to pre-blend certain components, such as the fragrance and the nonionic surfactant before adding such components into the surfactant mixture. In one embodiment, the surfactant mixture has a viscosity less than about 9,500 cps.

The self foaming agent is added to the composition and mixed after the surfactants are mixed and preferably after the optional ingredients are added thereto. If the mixing is performed in a non pressurized vessel, the surfactants and the self foaming agent are mixed using conventional mixing equipment with cooling. Cooling may be provided, for example, by utilizing a jacketed vessel and flowing cold brine through the jacket.

Although the self foaming agent may be added to the composition with mixing by any means known in the art, it is particularly useful to add the self foaming agent with mixing in a production instrument suitable for filling dispensing cans with products. The production instrument for filling dispensing cans typically has two feeds. Typically, one line feeds the self foaming agent to the instrument, while another line feeds the surfactant mixture and optional components to the instrument. Pistons are used to push the liquids into the instrument, creating a combined stream, which passes through static mixers to mix the two liquids, and then fills barrier packages. Commercially available dual piston fillers, such as those from Pamasol Company are suitable for these purposes. When these instruments are utilized, the process may be performed at ambient temperature.

As discussed above, the resulting composition should be in the form of a liquid crystal structure, i.e., a lamellar liquid crystalline phase. The liquid crystal has an oil droplet size less than 1 micron. It is a clear gel when stored in the product container and upon dispensing it from the can. The composition begins to foam upon contact with the skin, hair, or bath or shower water. The viscosity of the composition may range from at least about 20,000 cps to about 250,000 cps, e.g., from 20,000 cps to 150,000 cps and from about 20,000 cps to about 100,000 cps, as measured on a Brookfield viscometer. Preferably, no thickeners are added for purposes of increasing the viscosity of the composition, either before addition of the self foaming agent, or after addition of the self foaming agent.

The compositions of the present invention are preferably stored in aerosol containers, which are known in the art. Preferably, barrier packages are utilized. The barrier package may be selected from those known in the art, such as those possessing a first compartment for containing the self foaming agent along with the surfactant mixture and other optional components in the composition, as well as a second compartment or other means of enabling the discharge of the product from the first compartment via actuation of a valve by the user.

The barrier systems typically have a bag inside of a can. The bag is designed to be impermeable to the product stored in the can. The bag generally contains 3 layers; an outer polyester layer, a middle foil layer, and an inner polyethylene or polypropylene layer. Pressurized air is typically utilized as a propellant. The air pressure is generally from about 150 cm Hg to about 225 cm Hg, when the bag is empty. The ABS Advanced Barrier System™, available through CCL Container Company is particularly useful for the compositions of this invention. Another embodiment of a barrier package is the piston can, in which a moving piston in a cylindrical package tube separates the product zone from the pressurizing, dispensing-enabling zone.

The compositions of the present invention may be used to cleanse the body during a bath or a shower, as facial cleansers, and as shampoos. The compositions may be applied directly onto the desired body location with the hands, which is preferred, or may be applied via a fabric such as a washcloth, or through the use of a puff or loofah. Alternatively, the compositions may also be used to clean surfaces other the body, e.g. kitchen and bath counters, shower stalls, cars, upholstery, and the like.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

A 50 kg batch of a self foaming cleansing gel according to the invention was prepared. The materials used and process follows:

| Trade Name | INCI Name | % |
|---|---|---|
| Water | Aqua | 52.2 |
| Tegobetaine F50 | Cocamidopropyl Betaine (38% AM) | 12 |
| Texapon N70 | Sodium Laureth Sulfate (70% AM) | 20 |
| Plantacare 2000 | Decyl Glucoside | 8 |
| Sodium Benzoate | Sodium Benzoate | 0.5 |
| Glucamate DOE 120 | PEG-120 methyl glucose dioleate | 1.6 |
| Aminol N | PEG-4 Rapeseed amide | 1 |
| Propal | Isopropyl palmitate | 2 |
| Cremophor HR40 | PEG-40 hydrogenated castor oil | 1 |
| Neutrofoam | Perfume givaudan | 1 |
| EDTA | Tetra sodium EDTA | 0.1 |
| Sodium Citrate | Sodium Citrate | 0.3 |
| Citric Acid | Citric Acid | 0.3 |
| Total | | 100 |

To prepare the surfactant mixture, the water was heated to 50° C. EDTA, sodium citrate, and sodium benzoate were added to the water and mixed until dissolved. Texapon N70 (Cognis) was then added and mixed until dissolved. Glucamate DOE 120 (Americol) was then added and mixed until dispersed, then Tegobetaine F50 (Goldsmith) was added. Plantacare 2000 (Cognis) was added and mixed until dissolved. The batch was then cooled to 30° C. Isopropyl palmitate, Cremophor HR40 (BASF), and fragrance were then added. The pH was adjusted to 5.5 with citric acid. The final viscosity was 4,400 cps (Brookfield viscometer, spindle 3 @ 5RPM).

The surfactant mixture and the self foaming agent (75% isopentane/25% isobutane) were fed through a Pamasol dual piston can filler at a ratio of 92 parts by weight surfactant mixture to 8 parts by weight self foaming agent. The can was pressurized with air. The product was a clear, rather stiff gel, which dispensed from the package into the hand as a soft “mound” of product, which did not flow. When the product was spread on wet skin, the gel was easily sheared to be spread uniformly on the skin, and on so doing the gel was transformed to a creamy foam. On rinsing the foam was easily rinsed from the skin, leaving a soft and moisturized skin feeling.

### Example 2

| Trade Name | INCI Name | % |
|---|---|---|
| Water | Aqua | 45.8 |
| Tegobetaine F50 | Cocamidopropyl Betaine (38% AM) | 10.9 |
| Texapon N70 | Sodium Laureth Sulfate (70% AM) | 22.7 |
| Plantacare 2000 | Decyl Glucoside | 14.5 |
| Sodium Benzoate | Sodium Benzoate | 0.5 |
| Glycerine | Glycerine | 0.5 |
| Propal | Isopropyl palmitate | 1.8 |
| Cremophor RH40 | PEG-40 hydrogenated castor oil | 0.9 |
| Cosmedia Guar C261N | Guar Hydroxypropyl Trimonium | 0.2 |
| | Chloride | |
| | Fragrance | 0.9 |
| EDTA | Tetra sodium EDTA | 0.2 |
| Sodium Citrate | Sodium Citrate | 0.3 |
| Citric Acid | Citric Acid | 0.8 |
| Total | | 100 |

To a suitable mixing vessel equipped with a dispersator mixer were charged 400 g. deionized water, 3 g. Sodium Citrate, 5 g. glycerine, 3 g. Sodium Benzoate, and 2.6 g. tetrasodium EDTA. 250 g. of Texapon N-70 (Cognis) was added slowly to the vessel with mixing, and mixing continued until uniform. The prior premix was transferred into a suitable cosmetic mixing main tank with low intensity sweep mixing means. 120 g. Tegobetaine F50 (Goldschmidt) and 160 g. Plantaren 2000N (Cognix) were added to this main tank and mixed until uniform. In a separate vessel with propeller stirring means were combined 20 g.Ilsopropyl Palmitate,10 g. Cremophor RH40 (BASF), 2 g. Cosmedia Guar C261N (Cognis), and fragrance. This subphase was added to main tank and mixed until uniform. As needed, citric acid was added to achieve the pH target of 5.3-5.7, and suitable dyes were added. Sufficient water was added to make a totoal of 1000 g. Mixing was continued until the batch was uniform. The concentrate had a viscosity of about 3500 cps.

The concentrate described above was filled through a dual-piston filler together with 9.1% of a blend of 25% isobutane and 75% isopentane into bag-on-valve air-pressurized aluminum cans.

The product was a clear, rather stiff gel, which dispensed from the package into the hand as a soft “mound” of product, which did not flow. When the product was spread on wet skin, the gel was easily sheared to be spread uniformly on the skin, and on so doing the gel was transformed to a creamy foam. On rinsing, the foam was easily rinsed from the skin, leaving a soft and moisturized skin feeling.

### Example 3

| Trade Name | INCI Name | % |
|---|---|---|
| Water | Aqua | 67.6 |
| Kessco PEG 6000 DS | Polyethylene Glycol 6000 Distearate | 1.5 |
| Rhodapex ES-2X | Sodium Laureth (2) Sulfate (28% AM) | 10.0 |
| Tegobetaine L-7 | Cocamidopropyl Betaine (30% AM) | 8.0 |
| Monateric 1023 | Lauric-Myristic Phosphobetaine | 2.0 |
| | (30% AM) | |
| Atlas G-4280 | Polyoxyethylene (80) Sorbitan | 5.0 |
| | Monolaurate (72% AM) | |
| Fragrance | Fragrance | 0.2 |
| Versene 100XL | Tetrasodium EDTA Solution | 0.5 |
| Dowicil 200 | Quaternium-15 | 0.2 |
| Glycerine | Glycerine | 5.0 |
| Culinox #999 | Sodium Chloride | |
| Total | | 100 |

In a suitable vessel with propeller stirring means, 500 g of deionized water, 2 g. EDTA, and 50 g. glycerin were combined and heated to 65oC. 15 g. of Kessco PEG 6000 DS (Stepan) was added and the mixture was stirred until a uniform solution was achieved. Heating was discontinued and 100 g. Rhodapex ES-2K (Rhodia), 20 g. Monateric 1023 (Uniqema) , and 80 g. Tegobetaine L-7 (Goldschmidt) were added sequentially with stirring. When the temperature had decreased to 32° C., a premix of appropriate fragrance (2 g.) with 50 g of Atlas G-4280 (Uniqema) was added, together with preservative and colorant as desired. The pH was then adjusted with 20% sodium hydroxide to a target of pH 6.5. Sufficient water was added to make the total batch to 1000 g. The product was a clear liquid with a viscosity of 3300 cps.

When 91 parts of the above surfactant concentrate were combined with 9 parts of a 25/75 blend of isobutane with isopentane in a Coster pressure mixing vessel, and mixed, the result was a clear gel material. When dispensed, the product could be held in the hand as a clear gel portion, but when spread on wet skin the product spread easily and foamed spontaneously to give a luxurious, easy-spreading lather texture, which lasted through the cleansing process and then rinsed easily from the skin, leaving a clean, soft skin feel.

### Example 4

### Freeze Fracture Microscopy

The composition of Example 2 was subsequently examined using a freeze-fracture transmission electron microscope (FF-TEM). Two FF-TEM samples of duplicate formulation were prepared in accordance with techniques described in chapter 5 of “Low Temperature Microscopy and Analysis” by Patrick Echlin (1992). Briefly, each sample was cooled to 10° C. and then mounted between thin metal sheets and rapidly cooled with liquid propane to −196° C.

Each sample was then transferred under liquid nitrogen to a pre-cooled cold stage of a Balzers BAF-301 high vacuum freeze-etch unit (Techno Trade International, Lichtenstein). Each sample was fractured at low temperature and etched at −150° C. to remove a surface layer of water. The fracture faces were shadowed at an angle of 45° with platinum to create selective electron contrast. A thin layer of carbon was deposited over the entire fracture surface to create a continuous replica. The replicas were then examined using a JEOL 100CX2 electron microscope (Japanese Electronic Optical Laboratories, Japan).

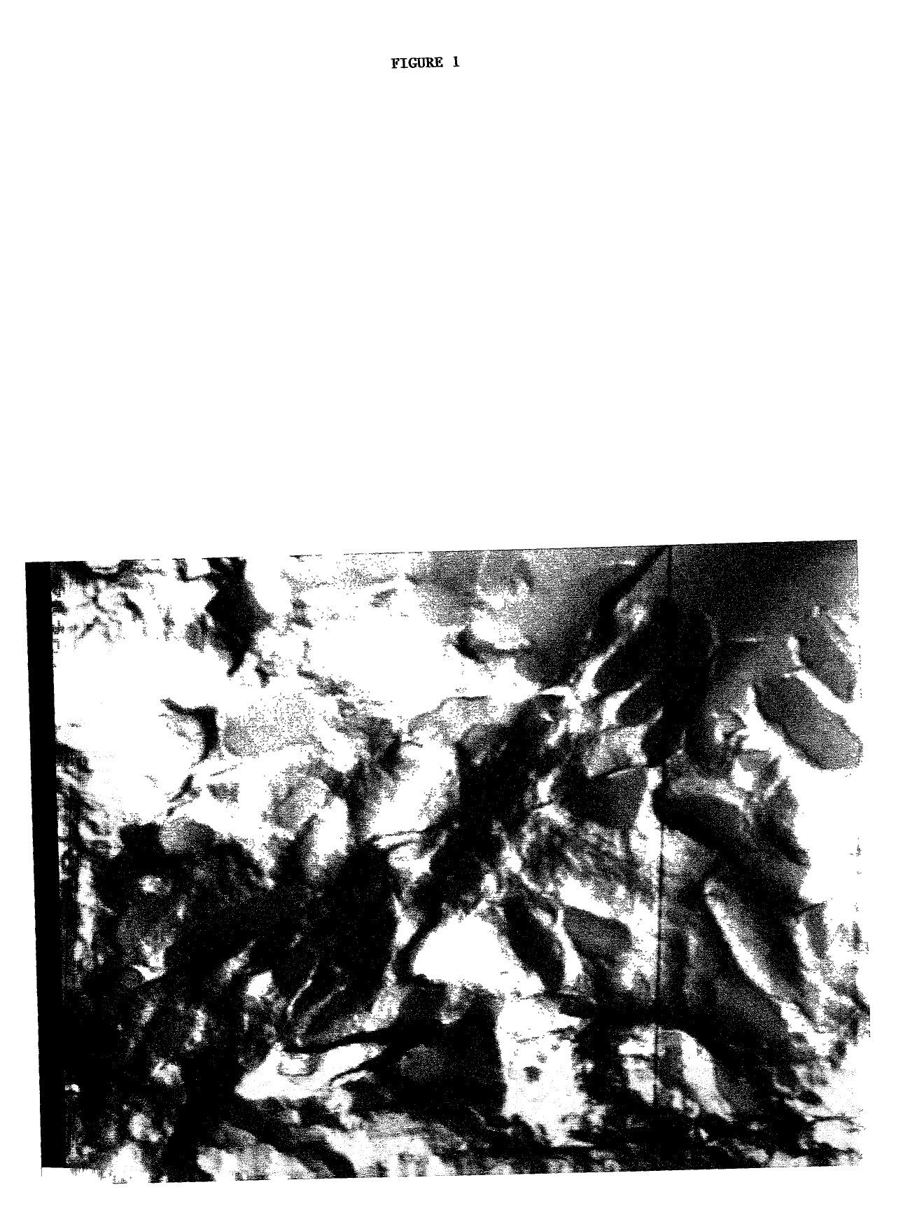

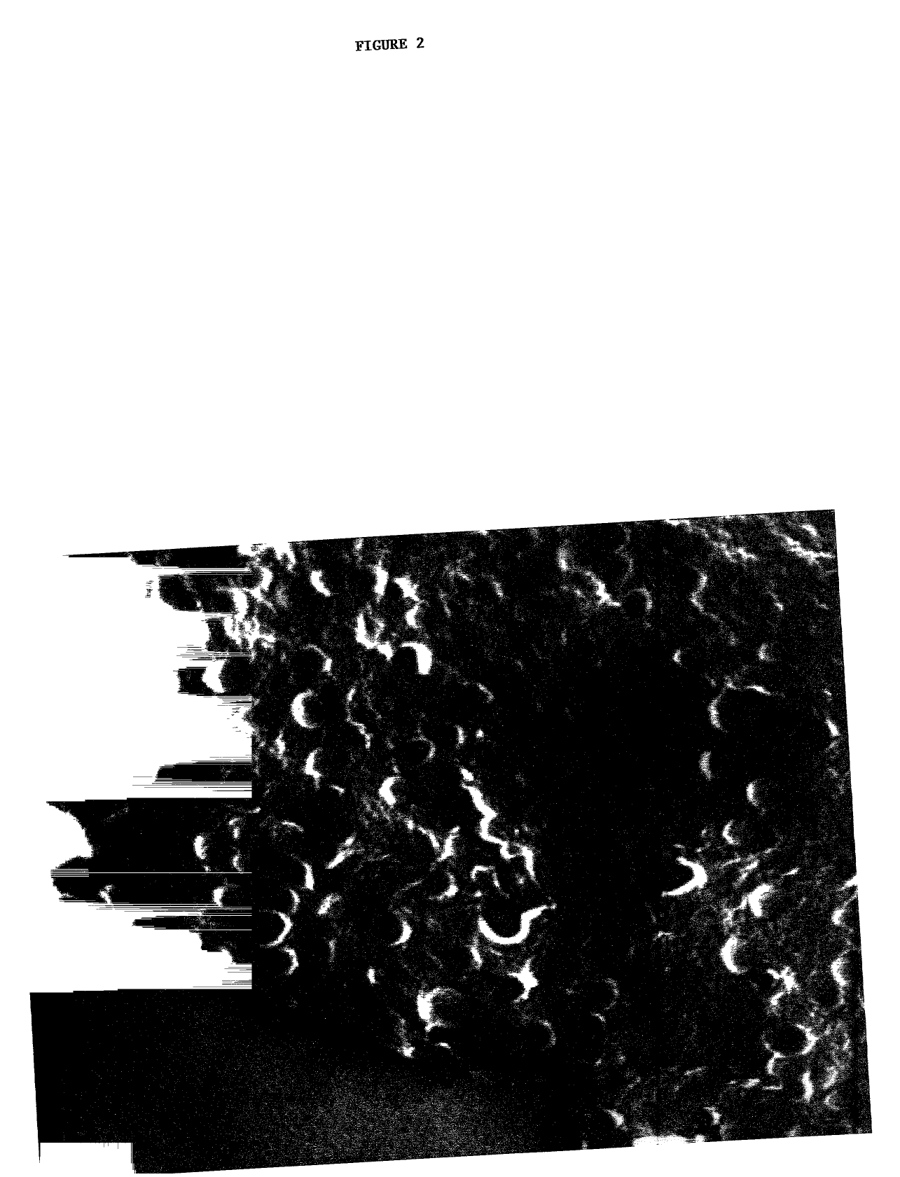

In FIG. 1 it is very evident that there are stacked liquid crystalline structure present in the electron micrographs of the formulation produced in Example 2. FIG. 2 also shows stacked liquid crystalline structures but in a cross sectional view of the formulation produced in Example 2.

### Example 5

Range finding studies were conducted wherein the following were varied: a) various surfactant combinations, b) varying amounts of surfactants, c) different self foaming agents, and d) varying amounts of self foaming agent.

The following samples were made in accordance with the procedure set forth in Examples 1-3 above:

1. 12 weight percent Tegobetaine F50; 38% active material (“AM”), 20 weight percent Texapon N70; 70% AM, 8 weight percent Plantacare 2000 UP; 30% AM, 1 weight percent Aminol N, 2 weight percent Estol 1517, 1 weight percent Glucamate DOE 120, 10 weight percent N-pentane

2. 12 weight percent Tegobetaine F50; 38% AM, 25 weight percent Texapon N70; 70% AM, 16 weight percent Plantacare 2000 UP; 30% AM, 2 weight percent Estol 1517, 1 weight percent Glucamate DOE 120, 0.2 weight percent Cosmedia Guar C261, 10 weight percent N-pentane

3. 12 weight percent Tegobetaine F50; 38% AM, 20 weight percent Texapon N70; 70% AM, 8 weight percent Plantacare 2000 UP; 30% AM, 1 weight percent Aminol N, 2 weight percent Estol 1517, 1.1 weight percent Glucamate DOE 120, 10 weight percent N-pentane

4. 15 weight percent Tegobetaine F50; 38% AM, 28 weight percent Texapon N70; 70% AM, 8 weight percent Plantacare 2000 UP; 30% AM, 2 weight percent Aminol N, 1.5 weight percent Estol 1517, 10 weight percent N-pentane

5. 15 weight percent Tegobetaine F50; 38% AM, 28 weight percent Texapon N70; 70% AM, 8 weight percent Plantacare 2000 UP; 30% AM, 1 weight percent Aminol N, 1.6 weight percent Estol 1517, 10 weight percent N-pentane

6. 12 weight percent Tegobetaine F50; 38% AM, 20 weight percent Texapon N70; 70% AM, 8 weight percent Plantacare 2000 UP; 30% AM, 1 weight percent Amino) N, 3 weight percent Atlas G1823, 2 weight percent Estol 1517,10 weight percent isopentane

7. 12 weight percent Tegobetaine F50; 38% AM, 25 weight percent Texapon N70; 70% AM, 8 weight percent Plantacare 2000 UP; 30% AM, 3 weight percent Arlatone SCL, 2 weight percent Estol 1517, 1 weight percent Glucamate DOE 120, 10 weight percent isopentane

8. 15 weight percent Tegobetaine F50; 38% AM, 28 weight percent Texapon N70; 70% AM, 8 weight percent Plantacare 2000 UP; 30% AM, 1 weight percent Aminol N, 10 weight percent N-pentane

9. 12 weight percent Tegobetaine F50; 38% AM, 25 weight percent Texapon N70; 70% AM, 16 weight percent Plantacare 2000 UP; 30% AM, 2 weight percent Estol 1517, 1 weight percent Glucamate DOE 120, 0.2 weight percent Cosmedia Guar C261, 10 weight percent isopentane

10. 12 weight percent Tegobetaine F50; 38% AM, 20 weight percent Texapon N70; 70% AM, 8 weight percent Plantacare 2000 UP; 30% AM, 1 weight percent Aminol N, 3 weight percent Ariatone SCL, 2 weight percent Estol 1517, 1 weight percent Glucamate DOE 120, 10 weight percent N-pentane

11. 12 weight percent Tegobetaine F50; 38% AM, 20 weight percent Texapon N70; 70% AM, 8 weight percent Plantacare 2000 UP; 30% AM, 1 weight percent Aminol N, 3 weight percent Arlatone SCL, 2 weight percent Estol 1517, 10 weight percent N-pentane

12. 12 weight percent Tegobetaine F50; 38% AM, 25 weight percent Texapon N70; 70% AM, 8 weight percent Plantacare 2000 UP; 30% AM, 1 weight percent Aminol N, 3 weight percent Arlatone SCL, 2 weight percent Estol 1517, 10 weight percent N-pentane

13. 12 weight percent Tegobetaine F50; 38% AM, 25 weight percent Texapon N70; 70% AM, 1 weight percent Aminol N, 3 weight percent Arlatone SCL, 2 weight percent Estol 1517, 1 weight percent Glucamate DOE 120, 10 weight percent N-pentane

14. 12 weight percent Tegobetaine F50; 38% AM, 25 weight percent Texapon N70; 70% AM, 1 weight percent Aminol N, 3 weight percent Arlatone SCL, 2 weight percent Estol 1517,10 weight percent N-pentane

15. 12 weight percent Tegobetaine F50; 38% AM, 20 weight percent Texapon N70; 70% AM, 8 weight percent Plantacare 2000 UP; 30% AM, 3 weight percent Aminol N, 3 weight percent Arlatone SCL, 1.5 weight percent Estol 1517, 0.5 weight percent Glucamate DOE 120, 10 weight percent N-pentane

16. 12 weight percent Tegobetaine F50; 38% AM, 20 weight percent Texapon N70; 70% AM, 8 weight percent Plantacare 2000 UP; 30% AM, 1 weight percent Aminol N, 3 weight percent Arlatone SCL, 2 weight percent Estol 1517, 0.5 weight percent Glucamate DOE 120, 0.3 weight percent Cosmedia Guar C261, 10 weight percent N-pentane

17. 12 weight percent Tegobetaine F50; 38% AM, 20 weight percent Texapon N70; 70% AM, 8 weight percent Plantacare 2000 UP; 30% AM, 1 weight percent Aminol N, 3 weight percent Arlatone SCL, 2 weight percent Estol 1517, 1 weight percent Glucamate DOE 120, 8 weight percent glycerine, 10 weight percent N-pentane

18. 12 weight percent Tegobetaine F50; 38% AM, 20 weight percent Texapon N70; 70% AM, 8 weight percent Plantacare 2000 UP; 30% AM, 1 weight percent Aminol N, 3 weight percent Arlatone SCL, 2 weight percent Estol 1517, 1 weight percent Glucamate DOE 120, 5 weight percent Lubrajel, 10 weight percent N-pentane

19. 15 weight percent Tegobetaine F50; 38% AM, 28 weight percent Texapon N70; 70% AM, 8 weight percent Plantacare 2000 UP; 30% AM, 2 weight percent Aminol N, 1.5 weight percent Estol 1517, 8 weight percent glycerine, 10 weight percent N-pentane

20. 12 weight percent Tegobetaine F50; 38% AM, 25 weight percent Texapon N70; 70% AM, 16 weight percent Plantacare 2000 UP; 30% AM, 1.6 weight percent Estol 1517, 0.2 weight percent Cosmedia Guar C261, 10 weight percent N-pentane

21. 15 weight percent Tegobetaine F50; 38% AM, 25 weight percent Texapon N70; 70% AM, 8 weight percent Plantacare 2000 UP; 30% AM, 2 weight percent Aminol N, 1.5 weight percent Estol 1517, 8 weight percent glycerine, 2 weight percent polysorbate 20, Givaudin 70/1, 10 weight percent N-pentane

22. 12 weight percent Tegobetaine F50; 38% AM, 25 weight percent Texapon N70; 70% AM, 8 weight percent Plantacare 2000 UP; 30% AM, 1 weight percent Aminol N, 2 weight percent Estol 1517, 8 weight percent glycerine, 10 weight percent N-pentane

23. 12 weight percent Tegobetaine F50; 38% AM, 25 weight percent Texapon N70; 70% AM, 1 weight percent Aminol N, 3 weight percent Arlatone SCL, 2 weight percent Estol 1517, 0.2 weight percent Glucamate DOE 120, 10 weight percent N-pentane

24. 15 weight percent Tegobetaine F50; 38% AM, 25 weight percent Texapon N70; 70% AM, 8 weight percent Plantacare 2000 UP; 30% AM, 2 weight percent Aminol N, 1.5 weight percent Estol 1517, 8 weight percent glycerine, 0.2 weight percent Methocel 40-202, 2 weight percent polysorbate 20, 10 weight percent N-pentane

25. 15 weight percent Tegobetaine F50; 38% AM, 25 weight percent Texapon N70; 70% AM, 8 weight percent Plantacare 2000 UP; 30% AM, 2 weight percent Aminol N, 1.5 weight percent Estol 1517, 8 weight percent glycerine, 0.1 weight percent Carbopol 940, 2 weight percent polysorbate 20, 10 weight percent N-pentane

26. 15 weight percent Tegobetaine F50; 38% AM, 25 weight percent Texapon N70; 70% AM, 8 weight percent Plantacare 2000 UP; 30% AM, 2 weight percent Aminol N, 1.5 weight percent Estol 1517, 8 weight percent glycerine, 2 weight percent polysorbate 20, 10 weight percent N-pentane

27. 12 weight percent Tegobetaine F50; 38% AM, 20 weight percent Texapon N70; 70% AM, 8 weight percent Plantacare 2000 UP; 30% AM, 2 weight percent Estol 1517, 1.5 weight percent Glucamate DOE 120

28. 12 weight percent Tegobetaine F50; 38% AM, 20 weight percent Texapon N70; 70% AM, 8 weight percent Plantacare 2000 UP; 30% AM, 2 weight percent Estol 1517,1.5 weight precent Glucamate DOE 120, 0.2 weight percent Cosmedia Guar C261

29. 12 weight percent Tegobetaine F50; 38% AM, 20 weight percent Texapon N70; 70% AM, 8 weight percent Plantacare 2000 UP; 30% AM, 2 weight percent Estol 1517, 1 weight percent Glucamate DOE 120

30. 12 weight percent Tegobetaine F50; 38% AM, 20 weight percent Texapon N70; 70% AM, 8 weight percent Plantacare 2000 UP; 30% AM, 1.5 weight percent Estol 1517, 1 weight percent Glucamate DOE 120

31. 12 weight percent Tegobetaine F50; 38% AM, 25 weight percent Texapon N70; 70% AM, 16 weight percent Plantacare 2000 UP; 30% AM, 2 weight percent Estol 1517, 0.75 weight percent Glucamate DOE 120

32. 12 weight percent Tegobetaine F50; 38% AM, 25 weight percent Texapon N70; 70% AM, 16 weight percent Plantacare 2000 UP; 30% AM, 2 weight percent Estol 1517, 0.75 weight percent Glucamate DOE 120, 0.2 weight percent Cosmedia Guar C261

### Example 6

The viscosity properties of some of the samples prepared in Example 5 above were tested. The viscosity of the surfactant mixture without the self foaming agent was measured using a Brokfield viscometer. Samples were stored in a water bath at 0° C. for 30 minutes, then the viscosity measured. The viscosities are reported in Table 1 below.

TABLE 1

| Sample | Percent Active Material | Viscosity |
|---|---|---|
| 2 | 27 | 13,800 |
| 5 | 28 | 10,000 |
| 9 | 27 | 13,800 |
| 10 | 21 | 30,500 |
| 11 | 21 | Too liquid |
| 12 | 25 | 6,630 |
| 13 | 22 | Too thick |
| 14 | 22 | 1,300 |
| 15 | 21 | 42,600 |
| 16 | 21 | 15,450 |
| 17 | 21 | 13,100 |
| 19 | 28 | 25,000 |
| 21 | 26 | 5,800 |
| 27 | 21 | 2,100 |
| 28 | 21 | 5,400 |

### Example 7

In this Example, the gel formation properties of some of the samples prepared in Example 5 above were tested. For each sample tested a jar was filled half way with the surfactant portions of the sample. The self foaming agent was then added to the jar and the jar was capped. The jar was shaken and the sample was observed to see if a gel formed. The results are reported in Table 2 below.

TABLE 2

| Sample | Gel Formation (pass/fail) |
|---|---|
| 1 | Pass (liquid gel) |
| 2 | Pass |
| 21 | Pass |
| 24 | Pass |
| 25 | Fail |
| 26 | Pass |

### Example 8

In this Example, the surfactant portions of some of the samples of Example 5 were combined with the self foaming agent portions of the samples in a Coster hand gel filler. The movement of the position was utilized to mix the samples. The samples were observed to see if a gel formed. The results are reported in Table 3 below.

TABLE 3

| Sample | Gel Formation (pass/fail) |
|---|---|
| 1 (10% N-pentane) | Fail |
| 1 (8% N-pentane) | Fail |
| 2 | Pass |
| 4 | Pass |
| 5 (10% isopentane) | Pass |
| 5 (8% isopentane) | Pass |
| 5 (6% isopentane) | Fail |
| 6 | Fail |
| 7 | Pass |
| 8 (10% isopentane) | Fail |
| 9 (20% isopentane) | Pass (thick gel) |
| 9 (15% isopentane) | Pass (thick gel) |
| 9 (10% isopentane) | Pass |
| 9 (7% isopentane) | Pass |
| 10 (20% N-pentane) | Pass (thick gel) |
| 10 (15% N-pentane) | Pass (thick) |
| 10 (10% N-pentane) | Pass (liquid gel) |
| 11 (20% N-pentane) | Pass (thick gel) |
| 11 (10% N-pentane) | Fail |
| 12 (20% N-pentane) | Pass (thick gel) |
| 12 (10% N-pentane) | Pass |
| 13 (20% N-pentane) | Pass (thick gel) |
| 13 (10% N-pentane) | Pass |
| 14 (20% N-pentane) | Pass (thin gel) |
| 14 (10% N-pentane) | Fail |
| 15 (20% N-pentane) | Pass (thin gel) |
| 15 (10% N-pentane) | Fail |
| 16 (20% N-pentane) | Pass (thin gel) |
| 16 (10% N-pentane) | Fail |
| 17 (20% N-pentane) | Pass (thin gel) |
| 17 (10% N-pentane) | Fail |
| 18 (20% N-pentane) | Pass (thin gel) |
| 18 (10% N-pentane) | Fail |
| 19 (20% N-pentane) | Pass (thin gel) |
| 19 (10% N-pentane) | Pass |
| 20 | Pass |
| 21 | Pass |
| 22 | Pass |
| 23 | Pass |
| 24 | Pass (thin gel) |
| 25 | Fail |
| 26 | Pass |
| 29 (15% isopentane) | Fail |
| 29 (10% isopentane) | Fail |

### Example 9

In this Example, some samples of Example 5 were filled into pressurized cans and dispensed to see if a self foaming gel was delivered from the pressurized can. A mixture of 75% isopentane and 25% isobutane was used as the self foaming agent. Unless otherwise indicated, 10% by weight self foaming agent mixture was combined with the surfactant mixture. The results are reported in Table 4 below.

TABLE 4

| Sample | Can Test (pass/fail) |
|---|---|
| 4 | Pass |
| 5 | Pass |
| 6 | Pass |
| 7 | Pass |
| 8 | Pass |
| 19 | Pass |
| 20 (13% self foaming agent) | Pass |
| 20 (8% self foaming agent) | Pass |
| 21 (13% self foaming agent) | Fail |
| 21 (8% self foaming agent) | Pass |
| 21 (7% self foaming agent) | Pass |
| 22 (13% self foaming agent) | Pass |
| 22 (8% self foaming agent) | Fail |
| 23 (8% self foaming agent) | Fail |
| 27 | Fail |
| 27 (8% self foaming agent) | Pass |
| 28 | Fail |
| 28 (8% self foaming agent) | Pass |
| 30 | Fail |
| 30 (8% self foaming agent) | Fail |
| 31 | Fail |
| 31 (8% self foaming agent) | Fail |
| 32 | Fail |
| 32 (8% self foaming agent) | Fail |

As demonstrated by the Examples above, only select surfactant compositions formed htelamellar phase as evidenced by gel formation upon mixing in the foaming agent. Also, come compositions failed to be suitable for commercial manufacture processes because of excessive sufactant mixture viscosity.

Having described the invention with reference to particular compositions, theories of effectiveness, and th like, it will be apparent to those of skill in the art that it is not intended that the invention be limited by such illustrative embodiments or mechanisms, and that modifications can be made without departing from the scope or spirit of the invention, as defined by the appended claims are meant to cover the claimed components and steps in any sequence which is effective to meet the objectives there intended, unless the context specifically indicates the contrary.

We claim:

1. A self foaming composition comprising:
(I) a surfactant mixture comprising (a) at least one anionic surfactant; (b) at least one amphoteric surfactant; and (c) optionally at least one nonionic surfactant; and
(II) at least one self foaming agent;
wherein the composition is in the form of a liquid crystalline structure and wherein the ratio of (a) to (b) to (c) is selected such that when the surfactant composition is mixed with the self foaming agent a gel consistency is obtained.

2. A self foaming composition according to claim 1, wherein the viscosity of the surfactant mixture prior to addition of the self foaming agent is less than about 9,500 cps.

3. A self foaming composition according to claim 2, wherein the viscosity of the composition after addition of the self foaming agent is at least about 20,000 cps.

4. A self foaming composition according to claim 1, wherein the concentration of the surfactant composition is at least about 10%, based on the total weight of the composition.

5. A self foaming composition according to claim 1, wherein the at least one anionic surfactant is present at from about 2% to about 30%, based on the total weight of the composition.

6. A self foaming composition according to claim 5, wherein the at least one anionic surfactant is present at from about 5% to about 20%, based on the total weight of the composition.

7. A self foaming composition according to claim 1, wherein the at least one anionic surfactant is selected from alkyl sulfates; alkyl ether sulfates; alkyl monoglyceryl ether sulfates; alkyl monoglyceride sulfates; alkyl monoglyceride sulfonates; alkyl sulfonates; alkylaryl sulfonates; alkyl sulfosuccinates; alkyl ether sulfosuccinates; alkyl sulfosuccinamates; alkyl amidosulfosuccinates; alkyl carboxylates; alkyl amidoethercarboxylates; alkyl succinates; fatty acyl sarcosinates; fatty acyl amino acids; fatty acyl taurates; fatty alkyl sulfoacetates; alkyl phosphates; alkyl ether phosphates; and mixtures thereof.

8. A self foaming composition according to claim 1, wherein the at least one anionic surfactant includes sodium laureth sulfate.

9. A self foaming composition according to claim 1, wherein the at least one amphoteric surfactant is present at from about 2% to about 20%, based on the total weight of the composition.

10. A self foaming composition according to claim 9, wherein the at least one amphoteric surfactant is present at from about 3% to about 15%, based on the total weight of the composition.

11. A self foaming composition according to claim 1, wherein the at least one amphoteric surfactant is selected from amphocarboxylates, alkyl betaines, amidoalkyl betaines, amidoalkyl sultaines, amphophosphates, phosphobetaines, pyrophosphobetaines, carboxyalkyl alkyl polyamines, alkyl amino monoacetates, alkyl amino diacetates, and mixtures thereof.

12. A self foaming composition according to claim 11, wherein the at least one amphoteric surfactant include cocamidopropyl betaine.

13. A self foaming composition according to claim 1, wherein the surfactant mixture includes from about 1% to about 15% of at least one nonionic surfactant, based upon the total weight of the composition.

14. A self foaming composition according to claim 13, wherein the at least one nonionic surfactant is selected from polyoxyethylene derivatives of polyol esters, ethylene oxide/propylene oxide copolymers, (poly)glycerol esters, (poly)glycerol fatty acids, fatty acid alkanolamides, alkoxylated monoalkanolamides, alkoxylated dialkanolamides, aminoxides, ethoylated fatty alcohols, ethoxylated fatty esters, ethoxylated glucosides, fatty gluconamides, and mixtures thereof.

15. A self foaming composition according to claim 13, wherein the at least one nonionic surfactant is selected from long chain alkyl glucosides or polyglucosides, which are the condensation products of (a) a long chain alcohol containing from about 6 to about 22 with (b) glucose or a glucose-containing polymer.

16. A self foaming composition according to claim 15, wherein the alkyl glucoside is selected from octyl glucoside, decyl glucoside, lauryl glucoside and mixtures thereof.

17. A self foaming composition according to claim 1, wherein the composition is free from ethoxylated fatty alcohols and ethoxylated fatty esters.

18. A self foaming composition according to claim 1, wherein the at least one self foaming agent is selected from pentane, isopentane, butane, isobutane, and mixtures thereof.

19. A self foaming composition according to claim 18, wherein the at least one self foaming agent comprises, based upon the total weight of self foaming agents, about 70% to about 90% isopentane and from about 10% to about 30% isobutane.

20. A self foaming composition according to claim 19, wherein the at least one self foaming agent comprising about 75% isopentane and about 25% isobutane, based upon the total weight of the self foaming agents.

21. A self foaming composition according to claim 1, wherein the at least one self foaming agent is present in the composition in an amount, based upon the total weight of the composition, of from about 4% to about 15%.

22. A self foaming composition according to claim 21, wherein the at least one self foaming agent is present in the composition in an amount, based upon the total weight of the composition, of from about 6% to about 12%.

23. A self foaming composition according to claim 1, having a pH in the range of from about 3 to about 9.

24. A self foaming composition according to claim 23, having a pH in the range of from about 4 to about 7.5.

25. A self foaming composition according to claim 1, wherein said composition is free from thickening agents or viscosity modifiers.

26. A self foaming composition according to claim 1, further comprising at least one conditioner.

27. A self foaming composition according to claim 26, wherein the at least one conditioner is selected from a cationic cellulose derivative; a cationic guar derivative; and derivatives and copolymers of diallyldimethylammonium chloride.

28. A self foaming composition according to claim 1, further comprising at least one skin conditioning agent.

29. A self foaming composition according to claim 28, wherein the at least one skin conditioning agent is selected from caprylic capric triglycerides, C₁₂-C₁₅ alcohol benzoates, isopropyl palmitate, and glycerine.

30. A self foaming composition according to claim 1 wherein the at least one anionic surfactant is sodium laureth sulfate, the at least one amphoteric surfactant is cocamidopropyl betaine and the at least one nonionic surfactant is decyl glucoside.

31. A self foaming composition according to claim 1, packaged in a barrier system.

32. A self foaming composition according to claim 31, wherein the barrier system is selected from a bag inside of a can or a piston system.

33. A self foaming composition according to claim 32, wherein the bag contains an outer polyester layer, a middle foil layer, and an inner polyethylene or polypropylene layer.

34. A process for making a self foaming composition, said process comprising the steps of (1) combining (a) at least one anionic surfactant; (b) at least one amphoteric surfactant;and (c) optionally at least one nonionic surfactant to form a surfactant mixture; (2) adding at least one self foaming agent to the surfactant mixture; wherein the composition is in the form of a liquid crystalline structure and wherein the ratio of (a) to (b) to (c) is selected such that when the surfactant composition is mixed with the self foaming agent the viscosity of the composition increases.

35. A process according to claim 34, wherein the viscosity of the surfactant mixture prior to addition of the self foaming agent is less than about 9,500 cps.

36. A process according to claim 35, wherein the viscosity of the composition after addition of the self foaming agent is at least about 20,000 cps.

37. A process according to claim 36, wherein the he viscosity of the composition after addition of the self foaming agent ranges from at least about 20,000 cps to about 250,000 cps.

38. A process for cleansing the skin or hair of a mammal comprising applying to said skin or hair an effective amount of the self foaming composition according to claim 1.
